(19) United States
(12) Patent Application Publication
(10) Pub. No.: US 2019/0071855 A1
(43) Pub. Date: Mar. 7, 2019

(54) LAVATORY SERVICE INDICATORS AND SANITATION

(71) Applicant: Richard James Andrews, Houston, TX (US)

(72) Inventor: Richard James Andrews, Houston, TX (US)

(21) Appl. No.: 15/968,949

(22) Filed: May 2, 2018

Related U.S. Application Data
(60) Provisional application No. 62/554,879, filed on Sep. 6, 2017.

(51) Int. Cl. E03D 9/00; A61L 2/18; A61L 9/05

(52) U.S. Cl. CPC E03D 9/005; A61L 2202/25; A61L 9/05; A61L 2/18

(57) ABSTRACT

Apparatus and methods are described that provide immediate and accurate indication of whether or not an aircraft or vessel lay has been utilized in operation of the aircraft or vessel via a dissolving use indicator placed in the lay. Using the lay use indicators provided herein, upon completion of a voyage and cursory inspection, lay service can be requested or declined.

### REFERENCE TO RELATED APPLICATIONS

This application claims priority based on U.S. Provisional Application Ser. No. 62/554,879 filed Sep. 6, 2017, which is incorporated herein by reference in its entirety.

### FIELD OF THE INVENTION

This invention relates generally to methods and apparatus relating to lavatory service, particularly in general aviation aircraft and marine vessels.

### BACKGROUND OF THE INVENTION

Without limiting the scope of the invention, its background is described in connection with existing lavatory service in aircraft and marine vessels. Although larger aircraft serving the public in delivery of scheduled air services will typically have plumbing systems complete with enclosed lavatories and flushing toilets, most private and charter operated jets and turboprops have lavatories that utilize liquid chemical or dry chemical solutions to flush waste. When these aircraft reach private FBO (fixed base operation) terminals, the lavatories will be serviced by line staff via lavatory (“lav”) trucks or carts that pump out the lay waste or the pilots have to remove the lav box below the seat and give it to the line service for cleaning and replacing the fluids. Typically the lavatory service will be conducted even if the lavatory has not been used because the crew will not be certain as to whether the lavatory has or has not been used. Unnecessary lav service is time consuming and expensive but heretofore no solution has been available that provides ready indication of the need for lay service. Similarly, in nautical applications it may be difficult or impossible to determine whether or not the lavatories have been used.

There exists a need in the art for a lavatory use indicator that provides a readily observable means of determining whether a dry lavatory has been used and requires service. Such is provided herein.

### SUMMARY OF THE INVENTION

Provided herein are a lavatory (“lav”) use indicators for use in dry lavatories (“lavs”) that allow visual determination that the lav has been used. In certain embodiments the lav use indicators, include at least one thin planar element dimensioned to be positioned in a lay such that the element will be contacted with a waste stream or a flushing solution upon use of the lay. The lay use indicator is water soluble and partially or completely disintegrates upon contact with a liquid.

In certain embodiments, the lay use indicator further includes one or more additives selected from: a sanitizing additive, a deodorizing additive and a fragrance additive. In certain embodiments, the lay use indicator is printed with one or more of: a water soluble nature of the element, a source for the element, advertising for vessel or vessel service providers, decorative features, and functions provided by the element including one or more of sanitizing, deodorizing and fragrance functions. In some embodiments the lay use indicator is formed as a packet that contains the one or more additives.

The water soluble lay use indicator is a printable paper and is comprised of cellulosic materials, starch materials, polyhydroxyacid materials and combinations thereof in certain embodiments.

Also provided are methods for servicing a dry lavatory in a transport vessel, including placing at least one lav use indicator in the dry lav, wherein the lav use indicator is water soluble and partially or completely dissolves when contacted with a fluid, monitoring a dissolution status of the lav use indicator at a completion of a voyage by the transport vessel; and servicing the lav if partial or complete dissolution of the lavatory use indicator occurs. In certain embodiments of the method, the dry lavatory is provided with a clear water flushing fluid and the lavatory use indicator comprises a sanitizing and/or deodorizing composition that dissolves in the clear water flushing fluid when the lavatory is utilized.

### DESCRIPTION OF THE DRAWINGS

For a more complete understanding of the present invention, including features and advantages, reference is now made to the detailed description of the invention along with the accompanying figures:

FIG. 1A illustrates placement of a use indicator according to one embodiment of the present invention. FIG. 1B illustrates the effect of fluids on the use indicator.

FIG. 2A illustrates another embodiment of a use indicator in position in a lavatory. FIG. 2B illustrates the effect of fluids on the use indicator.

FIG. 3A illustrates an embodiment of a use indicator including sanitizer according to one embodiment of the present invention. FIG. 3B illustrates a side view of a use indicator embodiment configured as a pillow containing internal sanitizer. FIG. 3C illustrates a side view of a use indicator embodiment configured as a sheet having a layer of sanitizer applied to a surface.

FIG. 4 illustrates another embodiment of a lay use indicator including a sanitizer according to one embodiment of the present invention.

### DETAILED DESCRIPTION OF THE INVENTION

Provided herein are methods and apparatus that enable immediate and accurate indication of whether or not a toilet has been utilized in operation of a transport vessel such as an aircraft, train, marine vessel, recreational vehicle or portable toilet. Using the use indicators provided herein, upon completion of a flight or voyage by the transport vessel and upon cursory inspection, the requirement for lay service can be readily determined and implemented. More particularly, lay use indicators are provided that remain intact in a transport vessel lay under normal environmental conditions of heat and humidity. However, when contacted with a fluid, the lay use indicator will partially or completely dissolve in a waste stream as well as when contacted with flushing fluids. A partially or completely dissolved lay use indicator will prompt service of the lay by emptying the waste receptacle of the lay. Where the lay use indicator is observed to be intact, costly lay service may be avoided. In certain embodiments, lay use indicators are provided that partially or completely dissolve when contacted with fluids and also release sanitizing and/or deodorizing compounds into waste and flushing fluids. In such cases, the provision of sanitizing and/or deodorizing compositions that are only added to a flushing fluid when flushing allows the flushing tank to be loaded with clear water that lacks additives.

In certain embodiments, lav use indicators are provided that also function to impart a pleasant fragrance to the environment of the lav. In certain embodiments the lav use indicators provided herein comprise a water soluble printable paper that provides a surface for printing one or more of: a water soluble nature of the product, a source for the product, advertising for vessel or vessel service providers, decorative elements, and functions provided by the product including sanitizing, deodorizing and imparting desirable fragrances.

To facilitate the understanding of this invention, and for the avoidance of doubt in construing the claims herein, a number of terms are defined below. Terms defined herein have meanings as commonly understood by a person of ordinary skill in the areas relevant to the present invention. The terminology used to describe specific embodiments of the invention does not delimit the invention, except as outlined in the claims.

The terms such as “a,” “an,” and “the” are not intended to refer to a singular entity unless explicitly so defined, but include the general class of which a specific example may be used for illustration. The use of the terms “a” or “an” when used in conjunction with “comprising” in the claims and/or the specification may mean “one” but may also be consistent with “one or more,” “at least one,” and/or “one or more than one.”

The use of the term “or” in the claims is used to mean “and/or” unless explicitly indicated to refer to alternatives as mutually exclusive. Thus, unless otherwise stated, the term “or” in a group of alternatives means “any one or combination of” the members of the group. Further, unless explicitly indicated to refer to alternatives as mutually exclusive, the phrase “A, B, and/or C” means embodiments having element A alone, element B alone, element C alone, or any combination of A, B, and C taken together.

Similarly, for the avoidance of doubt and unless otherwise explicitly indicated to refer to alternatives as mutually exclusive, the phrase “at least one of” when combined with a list of items, means a single item from the list or any combination of items in the list. For example, and unless otherwise defined, the phrase “at least one of A, B and C,” means “at least one from the group A, B, C, or any combination of A, B and C.” Thus, unless otherwise defined, the phrase requires one or more, and not necessarily not all, of the listed items.

The terms “comprising” (and any form thereof such as “comprise” and “comprises”), “having” (and any form thereof such as “have” and “has”), “including” (and any form thereof such as “includes” and “include”) or “containing” (and any form thereof such as “contains” and “contain”) are inclusive or open-ended and do not exclude additional, unrecited elements or method steps.

The term “effective” as used in the specification and claims, means adequate to provide or accomplish a desired, expected, or intended result.

The terms “about” or “approximately” are defined as being close to as understood by one of ordinary skill in the art, and in one non-limiting embodiment the terms are defined to be within 10%, within 5%, within 1%, and in certain aspects within 0.5%.

For purposes of the present invention, the term “lav” means a lavatory or toilet.

The following examples are included for the sake of completeness of disclosure and to illustrate the methods of making the compositions and composites of the present invention as well as to present certain characteristics of the methods and compositions. In no way are these examples intended to limit the scope or teaching of this disclosure.

### Example 1

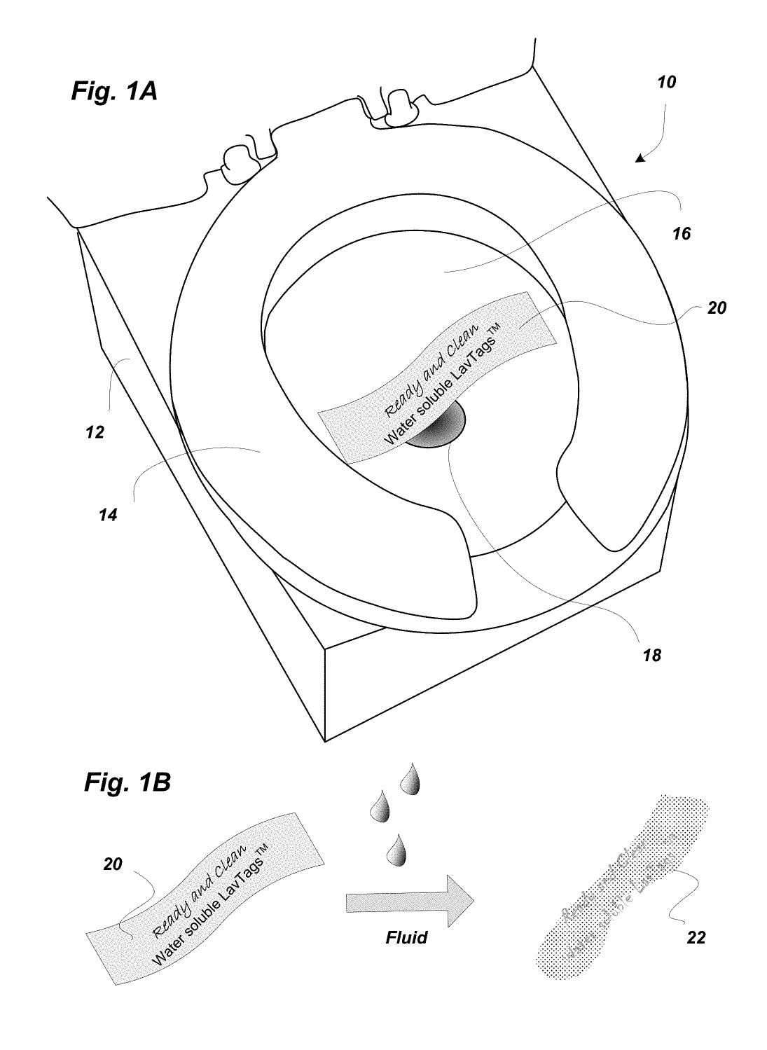

In one embodiment provided herein and depicted in FIG. 1A, a lav use indicator 20, referred to as a LAVTAG™, is shown in place in lav 10. Lav 10 will generally include a housing 12 that may include a fluid reservoir to provide flushing fluids, a pump and a waste reservoir, all of which are connected by tubing. The lav 10 will generally include a seat 14, a bowl 16 and a waste channel 18. Lav use indicator 20 is dimensioned for placement in bowl 16 such that any use of the lav will result in fluid interacting with the lav use indicator.

Lav use indicator 20 is composed partially or completely of a material that partially or completely dissolves when contacted with aqueous fluids. Thus, when fluid contacts the use indicator its integrity will be obviously impaired. In certain embodiments the use indicator is provided as a water soluble “paper” that will generally be formed of fibers that are formed together into a paper. By water soluble it is meant that those portions of the use indicator that are contacted with an aqueous fluid will partially or completely disclose within 20 minutes such that even a casual observation will reveal that an aqueous fluid has been in contact with the use indicator.

Any number of materials can be used to form the use indicator so long as that portion of the use indicator that contacts an aqueous fluid will: 1) partially or completely dissolve, 2) will not damage the lay, and 3) will be biodegradable and compatible with sewage waste disposal requirements.

Examples of such dissolving materials include cellulosic materials, starch materials, polyhydroxyacid materials, proteins, naturally occurring and synthetic polymers and combinations thereof.

Water dissolvable cellulosic materials include but are not limited to carboxymethyl cellulose, methyl cellulose, ethyl cellulose, hydroxypropyl cellulose, hydroxypropyl methyl cellulose, ethyl hydroxyethyl cellulose, hydroxypropyl cellulose gelatin and combinations thereof. Starches include but are not limited to natural and modified starches including methyl ether starch, aluminum starch octenyl succinate, high amylase starch, hydroxypropylated high amylose starch and hydroxy propyl starch phosphates.

Synthetic polymers include polyalkylene oxides, including polyethylene oxide and ethylene oxide/propylene oxide copolymers. Other synthetic polymers and copolymers thereof include but are not limited to methylmethacylate copolymer, carboxyvinyl polymer, polyquaternium polymers, polyacrylic acid, polymethacrylic acid, polymethacrylic acid copolymers, polyvinyl alcohol, poly (2-ethyl oxazoline), polyvinyl methyl ether, polyvinyl pyrrolidone/vinyl acetate copolymers, poly (n-isopropyl acrylamide), poly N-vinyl caprolactam, polyvinyl methyl oxazolidone, poly (2-isopropyl-2-oxazoline), and poly (2,4-dimethyl-6-triazinyl ethylene).

Naturally occurring polysaccharide polymers include xantham gum, guar gum, alginate, tragacanth gum, karaya gum (a.k.a. gum sterculia), acacia gum, arabic gum, locust bean gum, dextrin, pectin, chitin, chitosan, levan polysaccharide, elsinan, glucan, gellan gum, agarose, furcellaran, carrageenan, and pullulan (a.k.a. α-1,4-glucan; α-1,6-glucan). Proteins include collagen, zein, casein films and fibers, soy proteins and whey proteins. Examples of polyhydroxy acids include polyglycolide and copolymers thereof including (poly (lactic-co-glycolic acid), poly (glycolide-co-caprolactone) and poly (glycolide-co-trimethylene carbonate).

Preferably the use indicator will be printable with water based inks. In certain embodiments, printing on the use indicator provides certain instructions such as that the indicator is water soluble so that the user understands that the indicator need not and should not be removed from the lay during use. The printing may further contain indicia of origin and may provide an advertising surface for labelling.

### Example 2

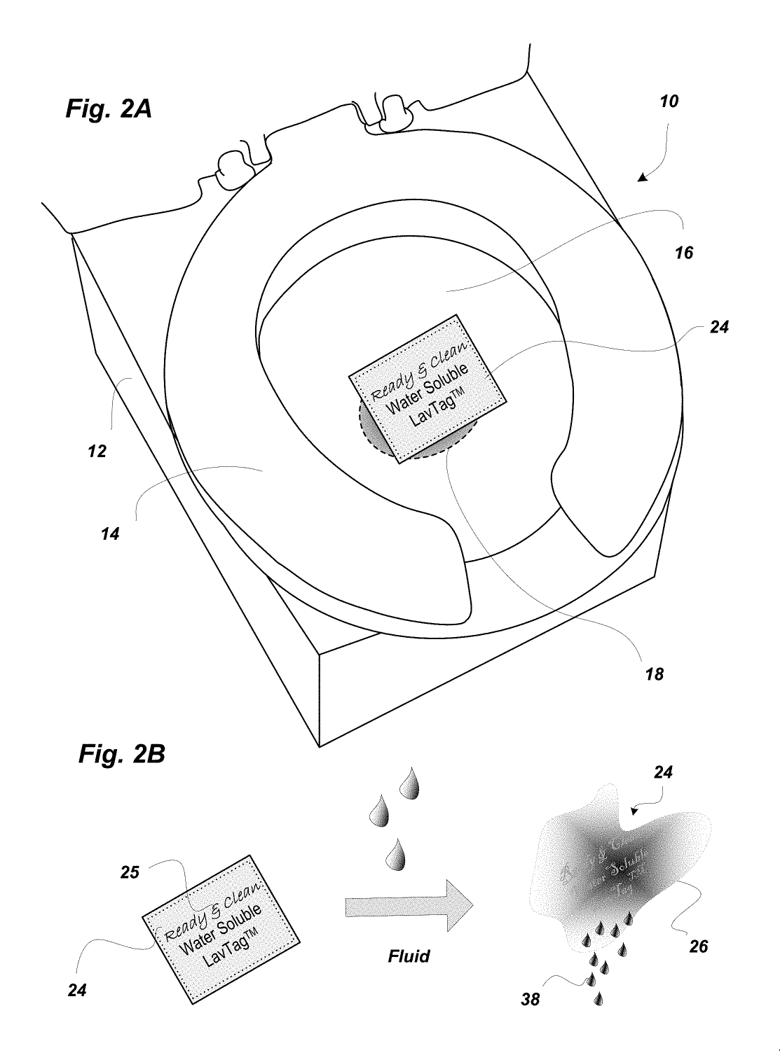

In another embodiment depicted in FIG. 2A, a lay use indicator 24 is provided that includes printing 25 and further includes one or more sanitizing, odor neutralizing and/or fragrance products. As used herein the term “sanitizing products” means products able to reduce the number of live microorganisms in a biological waste material or on a surface and for this reason sanitizing products are also known as “biocides.” As used herein the phrase “odor neutralizing” is used interchangeably with the term “deodorizing” and means reducing or removing objectionable odors. In certain cases a sanitizing product may also have odor neutralizing properties. For example agents such as sodium hypochlorite, hydrogen peroxide, potassium permanganate and ozone can oxidize most of the usual odor compounds and are thus deodorizing but may also kill live microorganisms and are thus sanitizing. Other sanitizing agents include chlorine, iodine and quaternary ammonium compounds.

As used herein fragrance products means products that impart a pleasant aroma. These may include essential oils. Certain fragrances that impart a pleasant aroma are also biocides and thus have sanitizing properties and deodorizing properties. For example, among the essential oils, the terpene carvacrol is present in the essential oil of Origanum vulgare (oregano), oil of thyme, and oils obtained from pepperwort and wild bergamot. Carvacrol inhibits growth of certain odor causing bacteria present in biological waste.

FIG. 2B shows the dissolved indicator 26 as a consequence of contacting an aqueous fluid. Loss or degradation of the integrity of printing 25 on the indicator provides further ready observation that the indicator has been contacted with a fluid and the lav has been used and should be serviced.

Typically aviation lays are filled with a flushing fluid that contains a sanitizing solution and/or neutralizing product. Sanitizing solutions may include one or more ammonium compounds including without limitation alkyl dimethyl benzyl ammonium chloride, and quaternary ammonium chlorides. Surfactants are often included. Surfactants of several classes may be utilized including anionic, cationic and zwitterionic surfactants, alkyl carboxylates (soaps) and non-ionic surfactants including long chain alcohols such as the fatty alcohols. Among the anionic surfactants, alkyl sulfates or sulfides including ammonium lauryl sulfate, sodium lauryl sulfate (SLS), sodium dodecyl sulfate (SDS), alkyl-ether sulfates such as sodium lauryl ether sulfate (SLES), sodium myreth sulfate, sodium dodecyl sulfide, and sodium dodecylbenezenesulfonate may be employed. Other materials including ethanol, sodium carbonates, ethylenediaminetetraacetic acid (EDTA), colorants and perfumes may be included.

Other biocidal materials include bromine-containing biocides such as 2,2-dibromo-3-nitrilopropionamide (DBNPA) and 2-bromo-2-nitropropane-1,3-diol (Bromopol), hypochlorite compounds such as sodium dichloro-s-triazinetrione and trichloro-s-triazinetrione, glutaraldehydes, isothiazolinones such as methylisothiazolinone (MIT), oxidants, copper alloys, and electrophiles including formaldehyde and formaldehyde-releasers.

Certain of the ingredients of lay sanitizing solutions including ammonium compounds and perfumes may result in residue buildup in the flush fluid and waste tanks. Because the flushing fluid typically includes sanitizing/deodorizing compounds, a relatively large amount of constituent chemicals require storage in the vessel tanks and must be disposed of.

In the embodiment depicted in FIG. 2A and FIG. 2B, lay indicator 24 includes sanitizing/deodorizing materials. The flushing fluid used in the lay can thus be clear water. Sanitizing/deodorizing materials 38 are only added to the system via dissolving of the lay indicator if the lay is used. Thus the amount of chemicals that must be used can be greatly reduced.

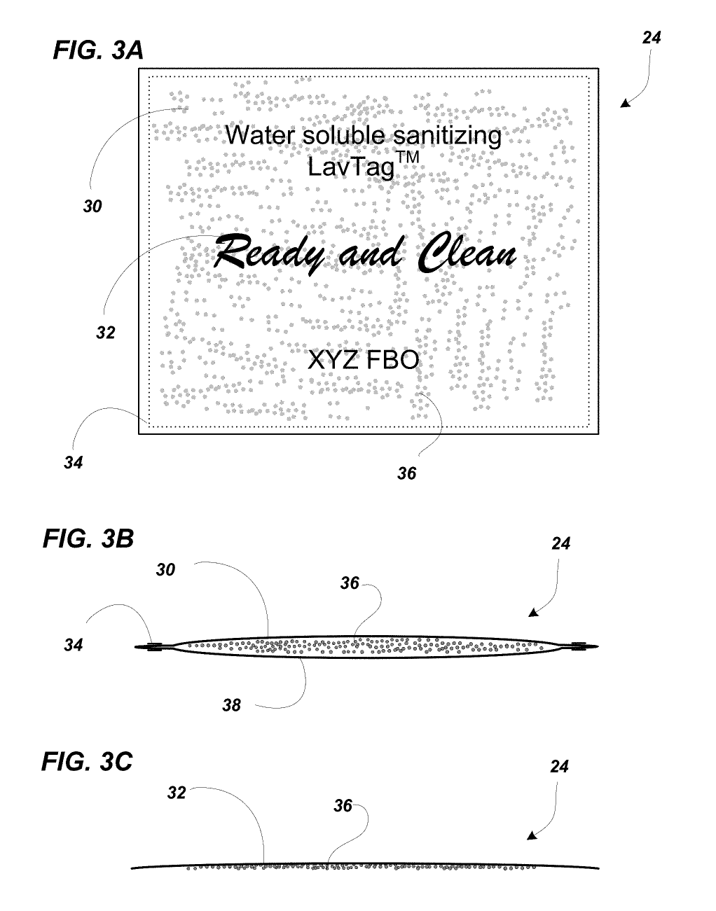

FIG. 3A illustrates a top surface of one embodiment of lay indicator 24 including sanitizing/deodorizing materials. Printing 32 on the use indicator provides certain instructions such as that the indicator is water soluble and provides sanitizing compounds so that the user understands that the indicator need not and should not be removed from the lay during use. The printing may further contain indicia of origin and may provide an advertising surface for labelling.

FIG. 3B illustrates a side view of one embodiment of a sanitizing lay use indicator 24 that is formed as a pouch or pillow having a top layer 30 and a bottom layer 38 both of which are formed of water soluble materials. The pouch or pillow formed by the top and bottom layers is filled with sanitizing and/or deodorizing compounds 36. In certain embodiments, the top layer 30 and the bottom layer 38 of the sanitary lay use indicator are formed of PVA materials and the edges 34 of the sanitizing lay use indicator 24 are sealed by heat during manufacture. In other embodiments the top layer 30 and the bottom layer 38 of the sanitary lay use indicator are formed of cellulosic, polyalkyl oxides or starches and the edges 34 of the sanitizing lay use indicator 24 are sealed by limited contact with an aqueous solution during manufacture.

FIG. 3C depicts a side view of an alternative embodiment where the sanitary lay use indicator is formed of a single sheet of water soluble material and the sanitizing/deodorizing compounds are applied to the water soluble sheet such as by spray coating.

### Example 3

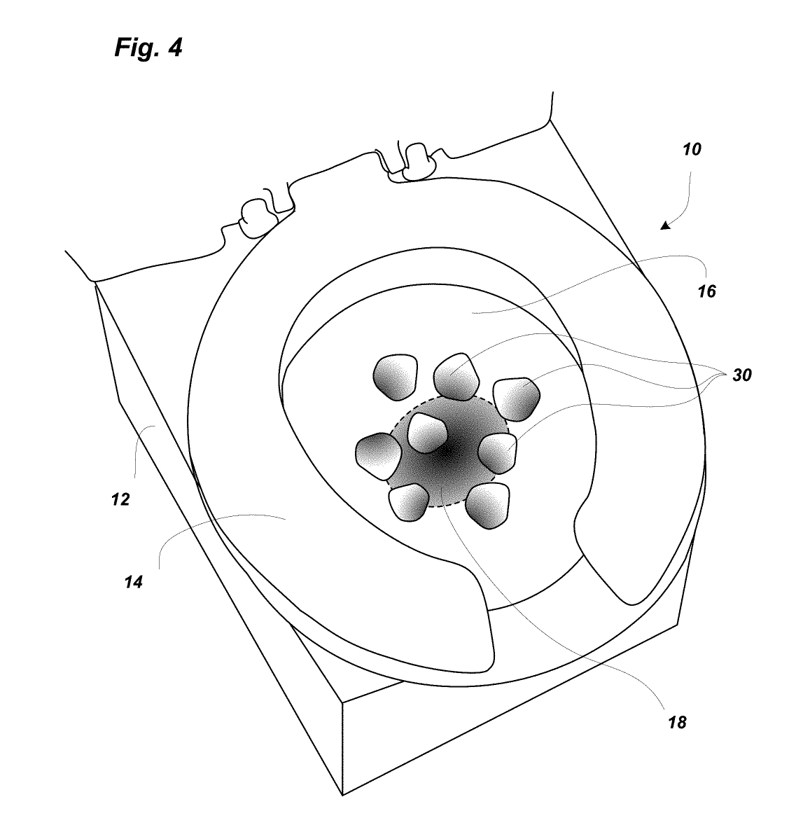

In another embodiment provided herein and depicted in FIG. 4, a plurality of lay use indicators 30 are shown in place in lay 10. Lay use indicators 30 are dimensioned for placement of a plurality of the indicators in bowl 16 such that any use of the lay will result in fluid interacting with at least one of the lay use indicators. The lay use indicators may be shaped in any geometric configuration such as dots, squares, rectangles, triangles or diamonds or may be shaped to emulate a flower or petal. The plurality of lay use indicators 30 may be rather quickly cast or scattered onto the lay as a further time saver. Lay use indicator 30 is composed partially or completely of a material that partially or completely dissolves when contacted with aqueous fluids. Thus, when fluid contacts one or more of the use indicators, the integrity of the contacted indicators will be obviously impaired. In certain embodiments the use indicator is provided as a water soluble “paper” that will generally be formed of fibers that are formed together into a paper. Lay use indicators 30 may further include one or more of at least one sanitizer, at least one deodorizer and at least one fragrance. The one or more sanitizer, deodorant and fragrance materials may be added to the water soluble paper prior to forming into the paper or may be deposited on the paper after manufacture.

For one non-limiting example, the lay use indicators 30 as depicted in FIG. 4 are shaped and colored to generally appear like rose petals that have been cast into the lay. In certain embodiments the lay use indicators are composed of a water soluble paper like material mixed with a sanitizer and an essential oil that imparts a desirable fragrance. Any use of the lay will cause one or more of the use indicators to completely or partially dissolve and signal use of the lay and a requirement for lay service at destination end.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A lavatory use indicator for use in a dry lavatory to allow visual determination that the lavatory has been used, comprising: at least one thin planar element dimensioned to be positioned in a lavatory such that the at least one element will be contacted with a waste stream or a flushing solution upon use of the lavatory, wherein the element is water soluble and partially or completely disintegrates upon contact with a liquid.

2. The lavatory use indicator of claim 1, wherein the thin planar element further comprises one or more additives selected from: a sanitizing additive, a surfactant, a biocide, a deodorizing additive and a fragrance additive.

3. The lavatory use indicator of claim 1, wherein at least one of the thin planar elements is printed with one or more of: a water soluble nature of the element, a source for the element, advertising for vessel or vessel service providers, decorative features, and functions provided by the element including one or more of sanitizing, deodorizing and fragrance functions.

4. The lavatory use indicator of claim 2, wherein the at least one thin planar element is formed as a packet that contains the one or more additives.

5. The lavatory use indicator of claim 1, wherein the water soluble element is comprised of cellulosic materials, starch materials, polyhydroxyacid materials, proteins, naturally occurring and synthetic polymers, and combinations thereof.

6. The lavatory use indicator of claim 5, wherein the cellulosic material is selected from carboxymethyl cellulose, methyl cellulose, ethyl cellulose, hydroxypropyl cellulose, hydroxypropyl methyl cellulose, ethyl hydroxyethyl cellulose, hydroxypropyl cellulose gelatin and combinations thereof.

7. The lavatory use indicator of claim 5, wherein the starch material is selected from methyl ether starch, aluminum starch octenyl succinate, high amylase starch, hydroxypropylated high amylose starch, hydroxy propyl starch phosphates, and combinations thereof.

8. The lavatory use indicator of claim 5, wherein the synthetic polymer is selected from polyalkylene oxides, polyethylene oxide, ethylene oxide/propylene oxide copolymers, methylmethacylate copolymer, carboxyvinyl polymer, polyquaternium polymers, polyacrylic acid, polymethacrylic acid, polymethacrylic acid copolymers, polyvinyl alcohol, poly (2-ethyl oxazoline), polyvinyl methyl ether, polyvinyl pyrrolidone/vinyl acetate copolymers, poly(n-isopropyl acrylamide), poly N-vinyl caprolactam, polyvinyl methyl oxazolidone, poly (2-isopropyl-2-oxazoline), poly (2,4-dimethyl-6-triazinyl ethylene), and combinations thereof.

9. The lavatory use indicator of claim 5, wherein the naturally occurring polymer is selected from xantham gum, guar gum, alginate, tragacanth gum, karaya gum, acacia gum, arabic gum, locust bean gum, dextrin, pectin, chitin, chitosan, levan polysaccharide, elsinan, glucan, gellan gum, agarose, furcellaran, carrageenan, pullulan, and combinations thereof.

10. The lavatory use indicator of claim 5, wherein the protein is selected from collagen, zein, casein films and fibers, soy proteins, whey proteins and combinations thereof.

11. The lavatory use indicator of claim 5, wherein the polyhydroxy acid is selected from polyglycolide and copolymers thereof including (poly (lactic-co-glycolic acid), poly (glycolide-co-caprolactone) and poly (glycolide-co-trimethylene carbonate).

12. The lavatory use indicator of claim 2, wherein the sanitizing additive is an ammonium compound.

13. The lavatory use indicator of claim 2, wherein the surfactant is selected from ammonium lauryl sulfate, sodium lauryl sulfate, sodium dodecyl sulfate, an alkyl-ether sulfates, sodium myreth sulfate, sodium dodecyl sulfide, sodium dodecylbenezenesulfonate and combinations thereof.

14. The lavatory use indicator of claim 2, wherein the biocide is selected from one or more of 2,2-dibromo-3-nitrilopropionamide, 2-bromo-2-nitropropane-1,3-diol, sodium dichloro-s-triazinetrione, trichloro-s-triazinetrione, glutaraldehydes, isothiazolinones, oxidants, copper alloys, formaldehyde, and formaldehyde-releasers.

15. The lavatory use indicator of claim 4, wherein the packet is formed by a top and a bottom layers filled with sanitizing and/or deodorizing compounds.

16. The lavatory use indicator of claim 15, wherein the top and bottom layers are formed of PVA materials and the top and bottom layers are sealed along their outer edges by heat during manufacture thereby forming the packet.

17. The lavatory use indicator of claim 15, wherein the top and bottom layers are formed of cellulosic, polyalkyl oxides or starch materials and the top and bottom layers are sealed along their outer edges by limited contact with an aqueous solution during manufacture thereby forming the packet.

18. A method for servicing a dry lavatory in a transport vessel, comprising:
placing at least one lavatory use indicator in the dry lavatory, wherein the lavatory use indicator is water soluble and partially or completely dissolves when contacted with a fluid;
monitoring a dissolution status of the at least one lavatory use indicator at a completion of a voyage by the transport vessel; and
servicing the lavatory if partial or complete dissolution of the lavatory use indicator occurs.

19. The method of claim 18, wherein the dry lavatory is provided with a clear water flushing fluid and the lavatory use indicator comprises a sanitizing and/or deodorizing composition that dissolves in the clear water flushing fluid when the lavatory is utilized.
